(19) United States
(12) Patent Application Publication
(10) Pub. No.: US 2022/0249342 A1
(43) Pub. Date: Aug. 11, 2022

(54) COSMETIC CLEANSING COMPOSITIONS WITH VISCOSITY MODIFIERS AND OILS

(71) Applicant: L'OREAL, Paris (FR)

(72) Inventors: Shumanta MITRA, Dublin, CA (US); Ryuji HARA, Westfield, NJ (US)

(73) Assignee: L'OREAL, Paris (FR)

(21) Appl. No.: 17/669,440

(22) Filed: Feb. 11, 2022

Related U.S. Application Data
(60) Provisional application No. 63/148,396, filed on Feb. 11, 2021.

(30) Foreign Application Priority Data
May 4, 2021 (FR) 2104701

(51) Int. Cl. A61K 8/37; A61K 8/36; A61K 8/81; A61K 8/02; A61K 8/92; A61Q 1/14; A61Q 19/10

(52) U.S. Cl. CPC A61K 8/37; A61K 8/361; A61K 8/8152; A61K 8/0208; A61K 2800/87; A61Q 1/14; A61Q 19/10; A61K 2800/33; A61K 8/922

(57) ABSTRACT

A cosmetic cleansing composition includes a cosmetic carrier system, and a cleansing coagulant system that includes at least one of each of an oil and a viscosity modifier, the viscosity modifier selected from synthetic (including petrochemical based) and natural polymers. The coagulant system can congeal and form a viscoelastic fluid to bind and remove sebum, makeup, dirt, pollution, dead skin and other unwanted material from skin.

### RELATED APPLICATIONS

This application claims priority to U.S. Patent Application No. 63/148,396 filed on Feb. 11, 2021, and French Application No. 2104701 filed May 4, 2021, each of which is titled COSMETIC CLEANING COMPOSITIONS WITH VISCOSITY MODIFIERS AND OIL, the entirety of which are incorporated herein by reference.

### FIELD OF THE INVENTION

The invention is related to novel cleansing and make-up remover compositions suitable for use on keratinous tissue such as skin and hair, the cosmetic cleansing composition containing a cleansing coagulant system that includes at least one of each of an oil and a viscosity modifier, the viscosity modifier selected from synthetic (including petrochemical based) and natural polymers.

### BACKGROUND OF THE INVENTION

Removal of stubborn makeup, sebum, dirt and skin debris is a challenge and a consumer pain point. This is particularly true for removal of mascara and waterproof mascara, long-wear foundation, and longer-lasting lip products. Typical cosmetic cleansers have varying effectiveness for removal of makeup, sebum, dirt and skin debris. Some oil-based cleansing and/or makeup removers may provide ease of makeup removal but leave behind an unpleasant and greasy skin feel. Less greasy alternatives can require tugging and rubbing on the skin and/or the delicate eye areas, causing irritation and leaving smudges and residue. And many cleansers and makeup removers include surfactants that can damage the keratinous tissue by denaturing protein, diminishing essential oils and peptides, leading to lipid irritation and a dry appearance.

Accordingly, there is a need for cosmetic cleansing and makeup removing compositions that are easy on the skin, efficiently remove makeup, sebum, dirt and skin debris without smudging and provide a soft and smooth, hydrated skin feel after use.

### BRIEF SUMMARY OF THE INVENTION

The summary is provided to introduce a selection of concepts in a simplified form that are further described below in the detailed description of the invention. This summary is not intended to identify key features of the claimed subject matter, nor is it intended to be used as an aid in determining the scope of the claimed subject matter.

In accordance with various embodiments, provided is a cosmetic cleansing composition that includes a cosmetic carrier system, and a cleansing coagulant system that includes at least one of each of an oil and a viscosity modifier, the viscosity modifier selected from synthetic (including petrochemical based) and natural polymers.

The cosmetic cleansing composition is easy on the skin, efficiently removes makeup, sebum, dirt and skin debris without smudging, and provides a soft and smooth skin feel after use. In some embodiments, the cosmetic cleansing composition is free from surfactants. In some embodiments, the cosmetic cleansing composition includes one or more surfactants.

In various embodiments, the disclosure provides a cosmetic cleansing composition comprising:
(a) cleansing coagulant system comprising:
   i. an oil component; and
   ii. a polymer component; and
(b) a cosmetic carrier system comprising one or more phases selected from the group consisting of a water phase, an oil phase, and combinations thereof.

The coagulant system can congeal and form a viscoelastic fluid to bind and remove sebum, makeup, dirt, pollution, dead skin and other unwanted material from skin. In some embodiments, the cleansing coagulant system that includes at least one of each of an oil and a viscosity modifier includes a natural based or food derived oil, such as linseed oil, and a methacrylate polymer components, such as poly(isobutyl methacrylate).

In some embodiments, the cleansing coagulant system is prepared as a reaction product of an oil component selected from the group consisting of glycerides, fatty acids, alkenes, and alkynes, with a polymer component, for example a synthetic polymer component such as a methacrylate or acrylate polymer component. In some embodiments, the cleansing coagulant system is prepared as a homogenous thermal reaction product. In some other embodiments, the reaction product may be produced using other reaction components that drive the formation of the reaction product that forms the cosmetic coagulant system.

In some embodiments, the oil and polymer components of the cleansing coagulant system can be applied to keratinous tissue, for example, on the skin, hair or nails, either in the form of a prepared cosmetic coagulant system or as a precursor that may be activated or reacted at the time of or just prior to application so as to form the cosmetic coagulant system on the surface of the keratinous tissue. According to such embodiments wherein the cosmetic coagulant system is activated and formed at the time of or just prior to application, the cleansing oil adsorbs sebum, skin dirt, particulate make up, etc. and reacts with the polymer to form a viscoelastic fluid that can be sloughed off. The cleansing coagulant system in the cosmetic carrier can be sloughed off, dissolved or washed-off in a natural solvent or water.

In some embodiments the cleansing coagulant system is formed just prior to or at the time of application to the keratinous tissue. In some embodiments, the cosmetic cleansing composition comprising the cleansing coagulant system forms as a film or mask on application that peels away in one piece or more than one piece from the skin.

In one or more embodiments, the cosmetic cleansing composition sets or dries into a film or mask which can be peeled away or sloughed off in one piece in less than 10 minutes.

In some embodiments, minimal or no water is needed for utilizing the cosmetic cleansing composition. Thus, in some embodiments, the cosmetic cleansing composition can be used with minimal water. In some embodiments, no water is required for use or removal of the cosmetic cleansing composition.

In some embodiments the cleansing coagulant system is included in the cosmetic cleansing composition in an activated form with or without a solvent. In such embodiments that include a solvent, the solvent may be present in either or both the cleansing coagulant system and the cosmetic carrier system.

In accordance with the various embodiments, the at least one oil in the cleansing coagulant system is selected from the group consisting of glycerides, fatty acids, alkenes, alkynes, and combinations thereof. In some embodiments, the at least one oil in the cleansing coagulant system is selected from the group consisting of sunflower oil, soybean oil, olive oil, corn oil, meadowfoam oil, and other natural oils. And in yet other embodiments, the at least one oil in the cleansing coagulant system is selected from silicone oils.

In accordance with the various embodiments, the at least one viscosity modifier in the cleansing coagulant system is selected from the group consisting of synthetic polymers, natural polymers, and combinations thereof. In some embodiments, the at least one viscosity modifier in the cleansing coagulant system is selected from the group consisting of petrochemical methacrylates, acrylates, allyl stearate/VA copolymer, and combinations thereof. In some embodiments, the at least one viscosity modifier in the cleansing coagulant system is selected from natural polymers selected from the group consisting of xanthan gum, guar gums, k-carrageenan, locust bean gum, alkyl dextrin, silica silicate, silica, montmorillonite, dibutyl ethylhexanoyl glutamide, dibutyl lauroyl glutamide, and combinations thereof.

In accordance with the various embodiments, the at least one oil in the cleansing coagulant system is present in a range from about 30% to about 45%, by weight, based on the weight of the cleansing coagulant system, and the at least one polymer is present in a range from about 0.1% to about 20%, by weight, based on the weight of the cleansing coagulant system.

In some embodiments, the cleansing coagulant system is present in the cosmetic cleansing composition in a range from about 1% to about 25%, by weight, based on the weight of the cosmetic cleansing composition, and the cosmetic carrier system is present in the range from about 75% to about 99%, by weight, based on the weight of the cosmetic cleansing composition.

In accordance with the various embodiments, the cosmetic cleansing composition includes a cosmetic carrier system comprising one or more phases selected from the group consisting of a water phase, an oil phase, and combinations thereof. In some embodiments, the cosmetic cleansing composition is essentially anhydrous and includes only any oily phase. In some embodiments, the cosmetic cleansing composition includes at least one water phase and at least one oily phase. In some embodiments, the cosmetic cleansing composition includes at least an oily/anhydrous phase.

In the various embodiments, the cosmetic carrier system includes one or more of components selected from the group consisting of oils/oily components and oil-phase solvents, water, water soluble solvents, surfactants, humectants, thickeners, preservatives, and other ingredients that may include actives, powders, fragrance oils, colorants, chelating agents, fragrances, and combinations thereof.

In some particular embodiments, the cosmetic carrier system includes an oily phase that includes one or more oily components selected from esters, for example, but not limited to, isopropyl myristate, or ethylhexyl palmitate, or a combination thereof, and ethers, for example, but not limited to, dicaprylyl ether, or other oily components and combinations thereof. In accordance with the various embodiments, the cosmetic cleansing composition has a consumer desirable fragrance and aesthetics.

In some embodiments, the formed cosmetic coagulant system includes a solvent. The solvent may be provided together with the cosmetic coagulant system or it may be provided in the cosmetic carrier system. The solvent comprises a cosmetically suitable solvent that is suitable to provide the cosmetic coagulant system at a viscosity that is desirable for use in the cosmetic cleansing composition. In some embodiments, the solvent comprises oil. In some embodiments, the solvent comprises glycerin, C1-4 alcohols, organic solvents, fatty alcohols, fatty ethers, fatty esters, polyols, glycols, vegetable oils, mineral oils, liposomes, laminar lipid materials, water, or any combinations thereof. In some embodiments, the solvent comprises a cosmetically acceptable organic solvent, which can be volatile or non-volatile compounds, and can include monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol, or any combinations thereof. In some embodiments, the solvent comprises a cosmetically acceptable organic solvent selected from ethylene glycol, propylene glycol, butylene glycol, hexylene glycol, propane diol, glycerin, or any combinations thereof.

In some embodiments, the disclosure provides articles of manufacture that include the cosmetic cleansing composition provided in a solid form, such as imbued in a pad or a wipe, or in a pod or a packet.

In some embodiments the cleansing coagulant system may be provided as a pad or a wipe wherein the cosmetic cleansing composition provided in a pad or wipe comprising a solid substrate that may formed from synthetic materials or formed from natural biodegradable and sustainably sourced natural originated fiber, natural fiber, or regenerated or recycled natural fiber. In some embodiments, the article of manufacture is a pad or a wipe infused with one or both of the cleansing coagulant system and the cosmetic carrier system.

In some embodiments, the disclosure provides articles of manufacture that include the cosmetic cleansing composition provided in at least one pod or packet that contains the cleansing coagulant system. In some such embodiments, the at least one pod or packet may contain both the cleansing coagulant system and the cosmetic carrier system. In some other embodiments, the at least one pod or packet may contain the cleansing coagulant system alone or with a solvent, and the article of manufacture includes at least one other article selected from a pod, packet or wipe that contains one of a cleansing coagulant system activator, the cosmetic carrier system, a cleansing coagulant system solvent, or a combination thereof.

In some particular embodiments, the cosmetic cleansing composition is an essentially anhydrous oil based composition that includes the cleansing coagulant system alone or with a solvent imbued or contained in an article comprising one of a pad, wipe, pod or packet, wherein the cosmetic carrier is either contained with the cleansing coagulant system or is separately imbued or contained in an article comprising one of a pad, wipe, pod or packet and comprises one of a cleansing coagulant system activator, a cleansing coagulant system solvent, one or more oils, one more ore oil-soluble components, or a combination thereof.

In use, when the cosmetic cleansing composition is applied to a keratinous tissue, for example skin or artificial skin that is soiled with one or more of sebum, makeup, or dirt and the % sebum, makeup or dirt extracted is quantified, significant sebum/dirt/oil containing makeup will be removed from the skin.

The cosmetic cleansing composition may be any suitable cosmetic cleansing composition, such as, but not limited to, an anhydrous cleansing oil, foaming cleanser, a cleansing milk, lotion or cream, an emulsion such as a water-in-oil or oil-in-water emulsion, or a multi-phase composition comprising at least one water phase, and is easy on the skin, efficiently removes makeup, sebum, dirt and skin debris without smudging, and provides a soft and smooth skin feel after use. The cosmetic cleansing composition in any of the product forms may be either a leave-on or a rinse-off formulation.

In some embodiments, the cosmetic cleansing composition is applied to keratinous tissue using an article comprising a solid substrate, for example comprising a disposable cotton or other type pad, a cloth, a brush or other type applicator.

In some embodiments, the cosmetic cleansing composition is applied to keratinous tissue in a generally liquidous form and thereupon forms into a solid or semi solid or film that may be peeled or sloughed off to remove makeup, sebum, soil and the like. In some embodiments, the cosmetic cleansing composition is applied in a series of steps from one or more articles of manufacture wherein after a first application one or more subsequent applications provides for reacting, fixing or coagulating the cleansing coagulant system component to form a semi solid or solid or congealed material that may thereafter be peeled or sloughed of washed form the keratinous tissue.

Other features and advantages of the present invention will be apparent from the following more detailed description, by way of example, the principles of the invention.

This disclosure describes particular embodiments in accordance with the general inventive concepts and is not intended to limit the scope of the invention in any way. Indeed, the invention as described in the specification is broader than and unlimited by the particular embodiments set forth herein, and the terms used herein have their full ordinary meaning.

### DETAILED DESCRIPTION OF THE INVENTION

The term “pad or wipe” is understood here to mean the combination composed of a water-insoluble support and a composition impregnated on the support. This pad or wipe can in particular be a wipe but it can also have any form including those described below. The water-insoluble substrate is absorbent and sufficiently strong not to disintegrate during the use thereof. The use of the pad or wipe as defined above for caring for the skin or hair and/or cleaning and/or removing makeup from and/or scrubbing the skin.

“Cosmetically acceptable” means a carrier that is compatible with any keratinous tissue.

“Keratinous tissue” includes but is not limited to skin, hair, and nails.

The term “surfactant free” means that excluded surfactants have not been added as a component. In some embodiments, a composition is devoid of surfactants. Those of skill in the art will appreciate that a surfactant may be present in a composition via its presence in one or more of the formulation components; thus, in some embodiments a composition may be “essentially surfactant-free” wherein surfactant is present at a concentration that does not exceed 5% by weight, and in some instances is present not more than 3% by weight, and in some instances is present not more than 1% by weight, based on the weight of the cosmetic cleansing composition. In some particular embodiments “surfactant-free” means that the cosmetic cleansing composition is free or devoid specifically of surfactant. Some specific but non-limiting examples of surfactants that are lacking from the cosmetic cleansing composition includes those with a C10-C20 fatty alcohol or acid hydrophobe condensed with from about 2 to about 100 moles of ethylene oxide or propylene oxide per mole of hydrophobe; C2-C10 alkyl phenols condensed with from 2 to 20 moles of alkylene oxide; mono- and di-fatty acid esters of ethylene glycol; fatty acid monoglyceride; sorbitan; mono- and di-C8-C20 fatty acids; polyoxyethylene sorbitan; alkyl polyglycosides and saccharide fatty amides (e.g. methyl gluconamides); alkyl ether sulfate and sulfonates; alkyl sulfates and sulfonates; alkylbenzene sulfonates; alkyl and dialkyl sulfosuccinates; C8-C20 acyl isethionates; C8-C20 alkyl ether phosphates; alkylethercarboxylate. Some specific surfactants that are lacking from the cosmetic cleansing composition include PEG-100 Stearate; PEG-20 Stearate and other esters of Poly(Ethylene Glycol); Sucrose Stearate and other emulsifiers based on sugar esters; Glyceryl Stearate and other glycerol esters; Disodium Ethylene Dicocamide PEG-15 Disulfate; Sodium Steroyl Glutamate and other fatty amides; Steareth-100 and other fatty ethers; Acrylates/C10-30 Alkyl Acrylate Crosspolymer and similar polymeric emulsifiers.

Cosmetic Cleansing Compositions

In accordance with the various embodiments, the cosmetic cleansing compositions include a cleansing coagulant system that includes an oil and a viscosity modifier selected from synthetic (including petrochemical based) and natural polymers. The coagulant system can congeal and form a viscoelastic fluid that can bind and remove any or all of sebum, makeup, dirt, pollution, dead skin and other unwanted material from skin. The cleansing coagulant system that includes viscosity modifiers includes food derived oil and methacrylate polymer components. The cosmetic cleansing and makeup removing compositions may be in the form of an anhydrous cleansing oil, foaming cleanser, a cleansing milk, lotion or cream, an emulsion such as a water-in-oil or oil-in-water emulsion, or a multi-phase composition comprising at least one water phase, and is easy on the skin, efficiently removes makeup, sebum, dirt and skin debris without smudging, and provides a soft and smooth skin feel after use.

Cleansing Coagulant System

In accordance with various embodiments, provided is a cosmetic cleansing composition that includes a cosmetic carrier system, and a cleansing coagulant system that includes at least one of each of an oil and a viscosity modifier, the viscosity modifier selected from synthetic (including petrochemical based) and natural polymers. The coagulant system can congeal and form a viscoelastic fluid to bind and remove sebum, makeup, dirt, pollution, dead skin and other unwanted material from skin. In some embodiments, the cleansing coagulant system that includes at least one of each of an oil and a viscosity modifier includes a natural based or food derived oil, such as linseed oil, and a methacrylate polymer components, such as poly(isobutyl methacrylate).

In accordance with the various embodiments, the cosmetic cleansing composition includes a cleansing coagulant system that includes a homogeneous thermal reaction product of an oil component selected from the group consisting of glycerides, fatty acids, alkenes, and alkynes, with a methacrylate or acrylate polymer component.

In various embodiments, the coagulant comprises a glyceride, such as, for example, the drying oil linseed oil, a polymer, such as, for example, poly(isobutyl methacrylate), and a cosmetically acceptable solvent, wherein the solvent may be present in one or the other of the cleansing coagulant system or the cosmetic carrier system, or both.

The cleansing coagulant system coagulates oil and oil-based materials such as sebum, and makeup independent of both agitation and temperature, and may be used in cosmetic carrier systems that are anhydrous, water based, including water that includes salt and fresh water.

In some particular embodiments, the cleansing coagulant system is prepared as a homogeneous thermal reaction product of an oil component selected from the group consisting of glycerides, fatty acids, alkenes, and alkynes, with a polymer component, for example a synthetic polymer component such as a methacrylate or acrylate polymer component. In some embodiments, the formed cosmetic coagulant system includes a cosmetically suitable solvent that is suitable to provide the cosmetic coagulant system at a viscosity that is desirable for use in the cosmetic cleansing composition.

In accordance with the various embodiments, the at least one oil in the cleansing coagulant system is selected from the group consisting of glycerides, fatty acids, alkenes, alkynes, and combinations thereof. In some embodiments, the at least one oil in the cleansing coagulant system is selected from the group consisting of sunflower oil, soybean oil, olive oil, corn oil, meadowfoam oil, and other natural oils. And in yet other embodiments, the at least one oil in the cleansing coagulant system is selected from silicone oils.

In accordance with the various embodiments, the at least one viscosity modifier in the cleansing coagulant system is selected from the group consisting of synthetic polymers, natural polymers, and combinations thereof. In some embodiments, the at least one viscosity modifier in the cleansing coagulant system is selected from the group consisting of petrochemical methacrylates, acrylates, allyl stearate/VA copolymer, and combinations thereof. In some embodiments, the at least one viscosity modifier in the cleansing coagulant system is selected from natural polymers selected from the group consisting of xanthan gum, guar gums, k-carrageenan, locust bean gum, alkyl dextrin, silica silicate, silica, montmorillonite, dibutyl ethylhexanoyl glutamide, dibutyl lauroyl glutamide, and combinations thereof.

In accordance with the various embodiments, the at least one oil in the cleansing coagulant system is present in a range from about 30% to about 45%, by weight, based on the weight of the cleansing coagulant system, and the at least one polymer is present in a range from about 0.1% to about 20%, by weight, based on the weight of the cleansing coagulant system.

In some embodiments, the cleansing coagulant system is present in the cosmetic cleansing composition in a range from about 1% to about 25%, by weight, based on the weight of the cosmetic cleansing composition, and the cosmetic carrier system is present in the range from about 75% to about 99%, by weight, based on the weight of the cosmetic cleansing composition.

In some embodiments, the cosmetic cleansing composition may be provided in the form of an anhydrous cleansing oil, foaming cleanser, a cleansing milk, lotion or cream, an emulsion such as a water-in-oil or oil-in-water emulsion, or a multi-phase composition comprising at least one water phase. In some embodiments, the cosmetic cleansing composition may be provided as an article of manufacture, for example in the form of a pad or wipe that is soaked with the cosmetic coagulant system or an unreacted precursor thereof. In yet other possible embodiments, the cosmetic cleansing composition may be provided in a pod or packet form, wherein the cosmetic coagulant system is encased in a pod casing, for example a water soluble film and the cosmetic carrier is provided within the pod or packet, or in a separate pod or packet. In some such embodiments, the cosmetic carrier is a solvent for cosmetic coagulant system.

The cleansing coagulant system may be provided, generally, according to the disclosure as set forth in U.S. Pat. No. 5,437,793, in which the invention is characterized as a coagulant product which comprises a glyceride such as linseed oil reacted with a polymer such as poly (isobutyl methacrylate) which is then diluted with a solvent, such as 2,2,4-trimethyl-1,3-pentanediol monoisobutyrate. According to that disclosure, the composition formed by the thermal reaction of the linseed oil with the isobutyl methacrylate polymer is a soft resinous product which, when diluted with a solvent, results in a mixture that in the teaching of the said patent can be sprayed onto an oil spill or otherwise introduced to the oil spill to coagulate the oil.

Additionally, as disclosed in the related U.S. Pat. No. 5,698,139 and additional patents cited, further experimentation led to the discovery of additional absorbent compositions produced from polymers and a variety of natural animal and vegetable oils, fatty acids, alkenes and alkynes, which absorbent compositions are all utilizable in preparing the filters of the present invention. More generally these latter compositions are the thermal reaction product of a polymer component with an oil component selected from the group consisting of glycerides, fatty acids, alkenes and alkynes. The reaction conditions can be adjusted to provide a “first endpoint” product or a “second endpoint” product. Preferred compositions are disclosed which comprise the thermal reaction products of methacrylate polymers with a glyceride derived from a variety of natural animal and vegetable oils, or the thermal reaction products of methacrylate polymers with a fatty acid or alkene or alkyne containing from about 8-24 carbon atoms. The combination of a methacrylate polymer component with any of these oil components can provide either a first or second endpoint product, depending upon the reaction conditions. The term “first endpoint product” is used to describe the solubility product of the reaction which is a cooperative structure held together by many reinforcing, noncovalent interactions, including Van Der Waals attractive forces. The term “second endpoint product” is used to describe the product of the reaction which is the result of covalent bond formation between the polymer component and the oil component, as indicated by the change in molecular weight.

Further according to U.S. Pat. No. 5,698,139, in a preferred embodiment, the prior art product is synthesized from an isobutyl methacrylate polymer, and the oil component is one derived from a natural oil, such as linseed oil or sunflower oil. Optionally, the composition is then diluted with a solvent, such as 2,2,4-trimethyl-1,3-pentanediol monoisobutyrate or acetone. The diluted composition can then be applied to a desired substrate for use as a filtration media. The polymer component of the said PA absorbent composition is a synthetic polymer such as polymers derived from methacrylates. Preferably, the polymer is derived from methyl methacrylate, ethyl methacrylate, isobutyl methacrylate, or n-butyl methacrylate, or may be a copolymer containing a methacrylate polymer. Most preferably, the polymer is a poly(isobutyl methacrylate) polymer such as that obtainable from ICI Acrylics as ELVACITE® 2045, or a methacrylate/methacrylic acid copolymer such as ELVACITE® 2008 or 2043. The test used to determine whether or not a polymer can be used in preparing the prior absorbent compositions is to combine the polymer component in question with the oil component, to see if the resultant combination forms a homogenous product after heating. It is stated in the patent disclosures that the polymer component percentage of the composition should range from about 15-75%, preferably 20-40%, or more preferably from about 25-35%, by weight.

Further according to U.S. Pat. No. 5,698,139, in one embodiment of the PA absorbent composition, the oil component of the composition is a glyceride derived from oils of vegetable or animal origin. Vegetable oils are obtained by cold pressing the seeds of a plant to obtain the oil contained therein. Of the vegetable oils, drying oils such as sunflower, tung, linseed, and the like; and semi-drying oils, such as soybean and cottonseed oil, have been shown to be useful as the glyceride component. Animal oils, such as, for example, fish oil, tallow and lard can also be used as a glyceride component of the composition. It is anticipated that any drying oil or semi-drying oil will work in the composition. Generally, a drying oil is defined as a spreadable liquid that will react with oxygen to form a comparatively dry film. Optionally, combinations of two or more glycerides can be used as reactants with the polymer to provide useful absorbent compositions. A glyceride derived from a drying oil, such as linseed oil, can be obtained from Cargill, Inc. as Supreme Linseed Oil, or sunflower oil. The glyceride should comprise from about 25-85%, preferably about 60-80%, and most preferably, from about 65-75% of the coagulant composition. All percentages in this disclosure are by weight, unless otherwise stated.

Further according to that disclosure, where the oil component of the prior composition is a fatty acid or alkene or alkyne utilized as the reactant with the polymer, it contains from about 8 to 24 carbon atoms, and preferably from about 10 to 22 carbon atoms. Such fatty acids, alkenes and alkynes are commercially available from many suppliers. Typical fatty acids include both saturated and unsaturated fatty acids, such as lauric acid [dodecanoic acid], linolenic acid, cis-5-dodecanoic acid, oleic acid, erucic acid [cis-docosanoic acid], 10-undecynoic acid, stearic acid, caprylic acid, caproic acid, capric acid [decanoic acid], palmitic acid, docosanoic acid, myristoleic acid [cis-9-tetradecenoic acid], and linoleic acid. Typical alkenes and alkynes contain at least one and preferably one or two degrees of unsaturation, and from about 8 to 24 carbon atoms, with 10-20 carbon atoms being preferred. Preferred alkenes and alkynes are those such as 1-decene, trans-5-decene, trans-7-tetradecene, 1,13-tetradecadiene, 1-tetradecene, 1-decyne, and 5,7-dodecadiyne. Further according to that disclosure, the PA absorbent composition is a product with characteristics different from either of the starting materials or a simple mixture of the two starting materials, thus showing that a new composition is produced by the thermal reaction. Specifically, the oil/polymer absorbent compositions pass a clear pill test after being heated at the elevated temperatures and do not separate into two parts upon being cooled but, rather form a homogenous, uniphase compound. The solvent can be selected from aliphatic hydrocarbons, aromatic hydrocarbons, alcohols, ketones, ethers, aldehydes, phenols, carboxylic acids, synthetic chemicals and naturally occurring substances.

It will be appreciated that while reference is made to the various patents and their disclosures as described above, the inclusion of this information is simply illustrative of some possible embodiments for providing a cleansing coagulant system according to the instant disclosure.

Further, it will be appreciated that the coagulant system produced according to the aforesaid disclosures, will include ingredients that are cosmetically acceptable and hence will not include polymers or solvents that would be toxic when applied to keratinous tissue.

It will be further understood that for purposes of the cosmetic cleansing composition disclosed herein, the cleansing coagulant system is formed with one or more oils and one or more viscosity modifiers in a cosmetic carrier system that is cosmetically acceptable. Accordingly, the above illustrated coagulant technology is not in any manner limiting and is not prescriptive of any of the components disclosed therein, including solvents, that may otherwise not be suitable for provide a cosmetically acceptable composition.

Carrier System

In the various embodiments of the cosmetic cleansing composition, the cosmetic carrier system includes at least one component. In some embodiments, the cosmetic carrier system includes at least one solvent for the cleansing coagulant system. In other embodiments, the cosmetic carrier system may include one or more other components, including one or more oily and/or water phases and components that are soluble therein.

Other Components

In various embodiments, the cosmetic cleansing composition may also comprise one or more of components selected from the group consisting of water, water soluble solvents, oils/oily components and oil-phase solvents, surfactants, thickeners, preservatives, powders, fragrance oils, colorants, surfactants, chelating agents, fragrances, actives, and combinations thereof.

It will be understood that additional ingredients, when present, are present in one or both of the cleansing coagulant system and the cosmetic carrier system and may include one or more oily and/or water phases and components that are soluble therein.

Oil Phase/Anhydrous Phase

In various embodiments, the cosmetic cleansing composition may include one or more phases that includes an oily/anhydrous phase. In accordance with the various embodiments, the amount of oily/anhydrous phase present in the cosmetic cleansing composition is in the range from about 10% to about 90%. The oily/anhydrous phase is characterized as being free or essentially free of water. In some embodiments, the cosmetic carrier system is anhydrous and does not include a water/aqueous phase.

In some particular embodiments, the cosmetic carrier system includes an oily phase that includes one or more oily components selected from esters, for example, but not limited to, isopropyl myristate, or ethylhexyl palmitate, or a combination thereof, and ethers, for example, but not limited to, dicaprylyl ether, or other oily components and combinations thereof.

Oils

In some embodiments, one or more oils may be present in the cosmetic cleansing composition. In accordance with some embodiments, oils may include one or more oils that comprise one or more fatty chain having a chain length from and including C8 to C24. In some embodiments, the oils are natural, food-derived oils, and in some particular embodiments, are oils of plant origin. Plant-derived oils include glyceride triesters, which are generally triesters of fatty acids and of glycerol, the fatty acids of which can have varied chain lengths from C4 to C24, it being possible for these chains to be saturated or unsaturated and linear or branched.

In some particular embodiments, the oils may be selected from olive oil, sweet almond oil, coconut oil, avocado oil, wheat germ oil, sunflower oil, grape seed oil, sesame oil, corn oil, apricot oil, castor oil, rhea oil, soybean oil, palm oil, rapeseed oil, cottonseed oil, hazelnut oil, macadamia oil, jojoba oil, alfalfa oil, poppy oil, pumpkin seed oil, marrow oil, blackcurrant oil, evening primrose oil, millet oil, barley oil, quinoa oil, rye oil, safflower oil, candlenut oil, passionflower oil, and musk rose oil. In some embodiments, the oils are selected from olive oil, sweet almond oil, coconut oil, avocado oil, and soybean oil.

In accordance with the various embodiments, each one of the oils when present may be present in the cosmetic cleansing composition at a concentration, by weight, of from about 1% to about 30%, or from about 3% to about 25%, or from about 5% to about 15%, or any suitable combination, sub-combination, range, or sub-range thereof by weight, based on the weight of the cosmetic cleansing composition.

In accordance with the various embodiments, the one or more oils, as combined, when present, are present in the cosmetic cleansing composition at a concentration, by weight, in an amount that is from about 15% to about 45%, or from about 25% to about 40%, or any suitable combination, sub-combination, range, or sub-range thereof by weight, based on the weight of the cosmetic cleansing composition. And in some particular embodiments, the oils, as combined, may be present in an amount that is at least about 25%, or at least about 30% by weight, based on the weight of the cosmetic cleansing composition. Thus, in some embodiments, the each one or combination of oils is present, by weight, based on the total weight of the cosmetic cleansing composition, from about at least 1, 2, 3, 4, 5, 6, 7, 8, 9, 10, 11, 12, 13, 14, 15, 16, 17, 18, 19, 20, 21, 22, 23 24, 25, 26, 27, 28, 29, 30, 31, 32, 22, 34 35 36, 37, 38, 39, 40, 41, 42, 43, 44, 45 to about 45 weight percent, including increments and ranges therein and there between.

In accordance with some embodiments, the cosmetic cleansing composition may include one or more branched or linear, liquid alkane with carbon chain length of C11 to C20. In various embodiments, liquid alkanes may be selected from those with a carbon chain length of from C11 to C20. The liquid alkanes may be selected from those with a carbon chain length of from C11 to C20, or from C15 to C19, or one of C11, C12, C13, C14, C15, C16, C17, C18 to C19. In some particular embodiments, suitable liquid alkanes that may be used according to the disclosure include hydrocarbon-based oils containing from 8 to 16 carbon atoms, and especially branched C8-C16 alkanes such as C8-C16 isoalkanes. In some exemplary embodiments, such liquid alkanes may be chosen from isoparaffins, for instance isododecane (also known as 2,2,4,4,6-pentamethylheptane), isodecane, and isohexadecane.

In some embodiments, the one or more branched or linear, liquid alkane, when present, comprises isohexadecane, C15-19 alkane, isododecane, undecane, tridecane and combinations thereof. In some embodiments the cosmetic cleansing composition may comprise two or more branched or linear, liquid alkanes.

The amount of each of the at least one branched or linear, liquid alkane, when present, is present in the cosmetic cleansing composition in a range of from about 1% to about 12% by weight, or from about 3% to about 11% by weight, or from about 5% to about 10% or any suitable combination, sub-combination, range, or sub-range thereof by weight, based on the weight of the cosmetic cleansing composition. One of ordinary skill in the art, however, will appreciate that other ranges are within the scope of the invention.

Thus, each of the at least one branched or linear, liquid alkane, when present in the cosmetic cleansing composition, may be present by weight, based on the total weight of the cosmetic cleansing composition, from about 1, 2, 3, 4, 5, 6, 7, 8, 9, to about 10 percent, including increments and ranges therein and there between.

In some embodiments, the cosmetic cleansing compositions may include one or more polar emollient. Emollients are oil-phase ingredients selected from esters, triglycerides, ethers, carbonates, alcohols, oils, butters, fatty acids, and their combinations thereof. In various embodiments, the polar emollients may be selected from those with a molecular weight of 400 g/mol or less. More, generally, the polar emollient may have a molecular weight in the range from about 50 g/mol to about 350 g/mol.

In some embodiments, suitable polar emollients that may be used according to the disclosure include those derived from C12-C50 fatty acids, preferably C16-C22 saturated fatty acids, and monohydric alcohols. In some embodiments, the polar emollients may be chosen from isopropyl myristate, methyl palmitate, isopropyl laurate, isopropyl palmitate, ethylhexyl palmitate, ethylhexyl laurate, ethylhexyl oleate, ethylhexyl isononanoate, myristyl myristate, 2-ethylhexyl caprate/caprylate (or octyl caprate/caprylate), 2-ethylhexyl palmitate, isostearyl neopentanoate, isononyl isononanoate, hexyl laurate, esters of lactic acid and of fatty alcohols comprising 12 or 13 carbon atoms, dicaprylyl carbonate and their mixtures.

In some embodiments, the one or more polar emollient may comprise one or more of isopropyl myristate, dicaprylyl ether, ethylhexyl palmitate, isopropyl palmitate, cetearyl ethylhexanoate, isononyl isononanoate, isopropyl isostearate, diisopropyl sebacate, coco caprylate/caprate, diisopropyl adipate, and combinations thereof. In some particular embodiments the one or more polar emollient includes isopropyl myristate. In some embodiments the cosmetic cleansing composition comprises two or more polar emollients. In some such embodiments, the polar emollient comprises isopropyl myristate and one or more additional polar emollients.

The amount of each of the at least one polar emollient, when present, is present in the cosmetic cleansing composition in a range of from about 1% to about 12% by weight, or from about 3% to about 11% by weight, or from about 5% to about 10% or any suitable combination, sub-combination, range, or sub-range thereof by weight, based on the weight of the cosmetic cleansing composition. One of ordinary skill in the art, however, will appreciate that other ranges are within the scope of the invention.

Thus, each of the at least one polar emollient, when present in the cosmetic cleansing composition, may be present by weight, based on the total weight of the cosmetic cleansing composition, from about 1, 2, 3, 4, 5, 6, 7, 8, 9, to about 10 percent, including increments and ranges therein and there between.

Water Phase/Aqueous Phase Solvents

In some embodiments, the cosmetic cleansing composition may be formed by providing a carrier system having one or more water phases that comprises water with or without one or more water-soluble solvents.

In accordance with some embodiments, the amount of water phase that may be present in the cosmetic cleansing composition is in the range from about 10% to about 90%.

Water

In accordance with some embodiments, water may be present in the cosmetic cleansing compositions in a range from about 1% to about 90%, or any suitable combination, sub-combination, range, or sub-range thereof by weight, based on the weight of the cosmetic cleansing composition. Thus, water is present, by weight, based on the total weight of the cosmetic cleansing composition, from about 1, 2, 3, 4, 5, 6, 7, 8, 9, 10, 11, 12, 13, 14, 15, 16, 17, 18, 19, 20, 21, 22, 23, 24, 25, 30, 35, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 75, 80, 85, to about 90 percent, by weight, including increments and ranges therein and there between. One of ordinary skill in the art, however, will appreciate that other ranges are within the scope of the invention.

The water used may be chosen from, for example, sterile demineralized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-Saunier, water from Eaux Bonnes, water from Rochefort, water from Saint Christau, water from Les Fumades, water from Tercis-les-Bains or water from Avene. The water phase may also comprise reconstituted thermal water, that is to say a water comprising trace elements such as zinc, copper, magnesium, etc., reconstituting the characteristics of a thermal water.

The pH of the cosmetic cleansing composition is not limited but is generally between 3.0 and 7.0. The pH can be adjusted to the desired value by addition of a base (organic or inorganic) to the cosmetic cleansing composition, for example ammonia or a primary, secondary or tertiary (poly)amine, such as monoethanolamine, diethanolamine, triethanolamine, isopropanolamine or 1,3-propanediamine, or alternatively by addition of an inorganic or organic acid, advantageously a carboxylic acid, such as, for example, citric acid. It will be understood by a skilled artisan that pH is a measurement of the hydrogen ion concentration in water which is determined by measuring the electrode potential using electrodes attached to a pH meter. It will be further understood that, due to the intrinsic variability in pH measurements, a skilled artisan would expect at least a 10% variability in pH measurements of compositions such as disclosed herein when measured in a typical laboratory by a typical skilled person using a typical pH meter.

Water-Soluble Solvents

In accordance with some embodiments, the cosmetic cleansing composition may include one or more water-soluble solvent. The term “water-soluble solvent” is interchangeable with the term “water-miscible solvent” and means a compound that is liquid at 25° C. and at atmospheric pressure (760 mmHg), and it has a solubility of at least 50% in water under these conditions. In some cases, the water-soluble solvent has a solubility of at least 60%, 70%, 80%, or 90% in water under these conditions. Non-limiting examples of water-soluble solvents include, for example, glycerin, alcohols (for example, C1-C30, C1-C15, C1-C10, or C1-C4 alcohols), polyols, glycols, and combinations thereof.

As examples of organic solvents, non-limiting mentions can be made of monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

Further non-limiting examples of water-soluble solvents include alkanols (polyhydric alcohols such as glycols and polyols) such as glycerin, 1,2,6-hexanetriol, trimethylolpropane, ethylene glycol, propylene glycol, diethylene glycol, butylene glycol, hexylene glycol, triethylene glycol, tetraethylene glycol, pentaethylene glycol, dipropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and combinations thereof.

In some cases, a water-soluble solvent may be selected from the group consisting of one or more glycols, C1-4 alcohols, and combinations thereof.

In accordance with the various embodiments total amount of the at least one water-soluble solvent, when present, may vary, is from about 0.5% to about 90%, or from about 0.5% to about 20%, or from about 1% to about 20%, or from about 1% to about 10%, or from about 2% to about 5% or any suitable combination, sub-combination, range, or sub-range thereof by weight, based on the weight of the cosmetic cleansing composition. One of ordinary skill in the art, however, will appreciate that other ranges are within the scope of the invention.

Thus, any one water-soluble solvent, when present, is present, by weight, based on the total weight of the cosmetic cleansing composition, from about 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 30, 40, 50, 60, 70, 80, to about 90 weight percent, including increments and ranges therein and there between.

It will be appreciated by a skilled artisan that any non-water solvents are present only to the extent and in amounts that do not materially adversely affect the basic and novel characteristic(s) of the claimed disclosure. Thus, in some embodiments that include preservatives, such preservatives will not materially adversely affect the cleansing coagulant system from coagulating to remove material from the keratinous tissue.

Surfactants

In some embodiments, the cosmetic cleansing composition may optionally include one or more surfactant.

The at least one surfactant may be an nonionic, cationic, anionic, or a zwitterionic surfactant. The at least one surfactant may be selected from the group consisting of polyoxyethylene sorbitan monolaureate, laureth-23, polyoxyethylated octyl phenol, 3-((3-cholamidopropyl) dimethylammonio)-1 propane sulfonate, sodium dilauramidoglutamide lysine, cholate, deoxycholate, sodium dodecylsulfate, TWEEN-80, and combinations thereof. The at least one surfactant may exclude esters. The at least one surfactant may be present in the cosmetic cleansing composition in a range from about 1% to about 5%, based on the weight of the cosmetic cleansing composition.

In some embodiments, the cosmetic cleansing composition may include one or more alkylated glycerol ester/surfactant. In some embodiments, the one or more surfactant may be selected from esters of polyols with fatty acids with a saturated or unsaturated chain containing for example from 8 to 24 carbon atoms (e.g., 12 to 22 carbon atoms), and alkoxylated derivatives thereof, such as glyceryl esters of a C8-C24 fatty acid or acids and alkoxylated derivatives thereof, polyethylene glycol esters of a C8-C24 fatty acid or acids and alkoxylated derivatives thereof, sorbitol esters of a C8-C24 fatty acid or acids and alkoxylated derivatives thereof, sugar (sucrose, glucose, alkylglucose) esters of a C8-C24 fatty acid or acids and alkoxylated derivatives thereof, ethers of fatty alcohols, ethers of sugar and a C8-C24 fatty alcohol or alcohols, and mixtures thereof.

In some embodiments the cosmetic cleansing composition may comprise at least one, and in some embodiments, two or more alkylated glycerol esters. In some such embodiments, the alkylated glycerol ester comprises one or more of PEG-7 glyceryl cocoate, PEG-7 caprylic/capric glycerides, PEG-6 caprylic/capric glycerides, disodium cocoyl glutamate (and) sodium cocoyl glutamate, coco-Betaine, caprylyl/capryl glucoside, Disodium cocoamphodiacetate, sodium cocoamphoacetate, polyglyceryl-4 caprate, and combinations thereof.

In some embodiments, each of the at least one alkylated glycerol ester in the cosmetic cleansing composition, when present, is present from about 0.1% to about 2%, or from about 0.2% to about 1.5%, or from about 0.5% to about 1%, or any suitable combination, sub-combination, range, or sub-range thereof by weight, based on the weight of the cosmetic cleansing composition. One of ordinary skill in the art, however, will appreciate that other ranges are within the scope of the invention.

In some particular embodiments, the total amount of alkylated glycerol ester/surfactant may be present up to about 2%. In some embodiments the total amount of alkylated glycerol ester/surfactant does not exceed about 2%. In some particular embodiments, the total amount of alkylated glycerol ester/surfactant is less than about 2%.

In various embodiments, the at least one surfactant, when present, may be present from about 1% to about 5% by weight of the cosmetic cleansing composition, and in some embodiments, from about 1% to about 2%, and in some embodiments, from about 3% to about 6%, and in some embodiments, from about 3% to about 4.5%, or any suitable combination, sub-combination, range, or sub-range thereof by weight, based on the weight of the cosmetic cleansing composition. In some embodiments, the cosmetic cleansing composition comprises more than one surfactant. One of ordinary skill in the art, however, will appreciate that other ranges are within the scope of the invention. Thus, in various embodiments, when present, each of the at least one surfactant may be present in a composition according to the disclosure from about 0.1, 0.2, 0.3, 0.4, 0.5, 0.55, 0.6, 0.7, 0.75, 0.8, 0.9, 1.0, 1.1, 1.2, 1.3, 1.4, 1.5, 1.6, 1.7, 1.8, 1.9, 2, 3, 4, to about 5 percent, by weight, including increments and ranges therein and there between.

Silicone Oils

The cosmetic cleansing composition described herein may contain one or more silicone oils.

As used herein, the term “silicone oil” is an oil (or non-aqueous medium) which is capable of evaporating on contact with the skin in less than one hour at ambient temperature and atmospheric pressure. The silicon oil is a cosmetic oil which is liquid at ambient temperature, having in particular a non-zero vapor pressure, at ambient temperature and atmospheric pressure, in particular having a vapor pressure ranging from 0.13 Pa to 40 000 Pa (10-3 to 300 mm Hg), and preferably ranging from 1.3 Pa to 13 000 Pa (0.01 to 100 mm Hg), and preferentially ranging from 1.3 Pa to 1300 Pa (0.01 to 10 mm Hg).

In some embodiments, the one or more silicone oils, may be, for example an hexamethyldisiloxane. Examples of silicone oils may be, for instance volatile linear or cyclic silicone oils, especially those with a viscosity of less than or equal to 8 centistokes (cSt) (8×10-6 m2/s, at 25° C.) and especially containing from 2 to 10 silicon atoms and in particular from 2 to 7 silicon atoms, these silicones optionally comprising alkyl or alkoxy groups containing from 1 to 10 carbon atoms. As silicone oils that may be used in the invention, mention may be made especially of dimethicones with viscosities of 5 and 6 cSt, octamethylcyclotetrasiloxane, decamethylcyclopentasiloxane, cyclohexasiloxane, dodecamethylcyclohexasiloxane, heptamethylhexyltrisiloxane, heptamethyloctyltrisiloxane, hexamethyldisiloxane, decamethyltetrasiloxane and dodecamethylpentasiloxane, and mixtures thereof.

The total amount of silicone oils, if present, may be present in an amount from about 0.1, 0.2, 0.3, 0.4, 0.5, 0.6, 0.7, 0.8, 0.95, 1, 1.1, 1.2, 1.4, 1.5, 1.6, 1.8, 2, 2.4, 2.6, 2.8, 3, 3.2, 3.4, 3.6, 3.8 to about 3.8, 4, 4.1, 4.2, 4.3, 4.4, 4.5, 4.6, 4.7, 4.8, 4.9, or 5 wt. of the total weight of the cleansing mask composition.

Humectant/Hydrating Agent

In some embodiments, one more humectants may be present in the cosmetic cleansing composition. In some embodiments, the humectant may comprise one or more of polyols, including, for example, glycerin, glycerol, glycols, such as caprylyl glycol, butylene glycol, propylene glycol, isoprene glycol, dipropylene glycol, hexylene glycol and polyethylene glycols, monoethylene glycol, diethylene glycol, triethylene glycol, diethylene glycol, hexylene glycol, glycol ethers such as monopropylene, dipropylene and tripropylene glycol alkyl(C1-C4)ethers, squalane, triacetin, sugars, such as glucose, xylitol, maltitol, sorbitol, sucrose pentaerythritol, inositol, pyrrolidone carboxylic acid, lactic acid, lithium chloride, acetamide MEA, sodium lactate, urea, dicyanamide, hyaluronic acid, aloe vera, honey, and seaweed extract.

In accordance with the various embodiments, when present, the amount of humectant present in the cosmetic cleansing composition can range from about 1% to about 10%, or from about 1% to about 8%, or from about 1% to about 5%, or from about 2% to about 3%, or any suitable combination, sub-combination, range, or sub-range thereof by weight, based on the weight of the cosmetic cleansing composition. One of ordinary skill in the art, however, will appreciate that other ranges are within the scope of the invention.

Thus, any one of or a combination of humectant may be present, by weight, based on the total weight of the cosmetic cleansing composition, is from about 1, 2, 3, 4, 5, 6, 7, 8, 9, to about 10 weight percent, including increments and ranges therein and there between.

Polymeric Thickeners

In accordance with some embodiments, the cosmetic cleansing composition may include one or more polymeric thickeners. In some embodiments, the one or more polymeric thickeners is in the anhydrous phase.

In some embodiments, the one or more thickener may be selected from one or more of natural polymers, for example starches (corn, rice, tapioca, potato), gums (xanthan carrageenan, gellan, sclerotium, tarabiotech fermentation). In some embodiments, the polymeric thickener can include one or more of cellulose-based thickeners (e.g., hydroxyethyl cellulose, hydroxypropyl cellulose, carboxymethylcellulose, cationic cellulose ether derivatives, quaternized cellulose derivatives, etc.), gums such as gums of microbial origin (e.g., xanthan gum, scleroglucan gum, etc.), and gums derived from plant exudates, including but not limited to guar gum and its derivatives (e.g., hydroxypropyl guar, cationic guar derivatives, etc.), gum arabic, ghatti gum, karaya gum, gum tragacanth, carrageenan gum, agar gum and carob gum, pectins, alginates, and starches. In some embodiments, the one or more thickeners is selected from gums. In some embodiments, the polymeric thickener may be one of carbomer, acrylates/C10-30 alkyl acrylate crosspolymer, polyacrylate crosspolymer-6, and combinations thereof.

The amount of each of the at least one polymeric thickener, when present, may be present in the cosmetic cleansing composition in a range of from about 0.01% to about 2%, or from about 0.01% to about 1.5%, or from about 0.3% to about 1.2%, or any suitable combination, sub-combination, range, or sub-range thereof by weight, based on the weight of the cosmetic cleansing composition. One of ordinary skill in the art, however, will appreciate that other ranges are within the scope of the invention.

In some embodiments, the total amount of polymeric thickener that may be present in the cosmetic cleansing composition is present from about 0.01% to about 5%, or from about 0.02% to about 2%, or from about 0.03% to about 1.5%, or from about 0.1% to about 0.2%, or any suitable combination, sub-combination, range, or sub-range thereof by weight, based on the weight of the cosmetic cleansing composition. One of ordinary skill in the art, however, will appreciate that other ranges are within the scope of the invention.

Thus, one or more polymeric thickener may be present by weight, based on the total weight of the cosmetic cleansing composition, from about 0.01, 0.02, 0.03, 0.04, 0.05, 0.06, 0.07, 0.08, 0.09, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5 to about 5.0 percent, including increments and ranges therein and there between.

Preservatives

In some embodiments, one or more preservatives and/or antimicrobials may be present in the cosmetic cleansing composition. Any preservative commonly used in cosmetic formulations is an acceptable preservative for the cosmetic cleansing compositions herein, such as phenoxyethanol, members from the paraben family such as the methyl, ethyl, propyl, butyl or isobutyl parabens, 4-hydroxy benzoic acid, benzoic acid, sorbic acid, dehydroacetic acid, triclosan, benzyl alcohol, chlorophenesin, or salicylic acid, for example. At more concentrated amounts of suitable solvents for optional additives, in particular, suitable solvents for antimicrobials and preservatives, members from the paraben family may be used as a preservative.

In some embodiments, the preservative may comprise one or more of preservatives selected from the group consisting of organic acids, parabens, formaldehyde donors, phenol derivatives, quaternary ammoniums, alcohols, isothiazolinones, and combinations thereof. Preservatives having antibacterial activity are optionally present in the cosmetic cleansing compositions of the present invention. Examples of organic acid preservatives include, but are not limited to, sodium benzoate, potassium sorbate, benzoic acid and dehydroacetic acid, sorbic acid, and combinations thereof. A preferred organic acid preservative system includes a mixture of sodium benzoate and potassium sorbate. Examples of paraben preservatives include, but are not limited to, alkyl para-hydroxybenzoates, wherein the alkyl radical has from 1, 2, 3, 4, 5 or 6 carbon atoms and for example, from 1 to 4 carbon atoms e.g., methyl para-hydroxybenzoate (methylparaben), ethyl para-hydroxybenzoate (ethylparaben), propyl para-hydroxybenzoate (propylparaben), butyl para-hydroxybenzoate (butylparaben) and isobutyl para-hydroxybenzoate (isobutylparaben). Examples of formaldehyde donor preservatives include, but are not limited to, 1,3-Dimethylol-5,5-dimethylhydantoin (DMDM hydantoin), imidazolidinyl urea, gluteraldehyde, and combinations thereof. Examples of quaternary ammonium preservatives include, but are not limited to, benzalkonium chloride, methene ammonium chloride, benzethonium chloride, and combinations thereof. Examples of alcohol preservatives include, but are not limited to, ethanol, benzyl alcohol, dichlorobenzyl alcohol, phenoxyethanol, and combinations thereof. Examples of isothiazolone preservatives include, but are not limited to, methylchloroisothiazolinone, methylisothiazolinone, and combinations thereof.

Other suitable preservatives include, but are not limited to, chloroacetamide, triclosan and iodopropynyl butylcarbamate, pyridine derivatives (e.g., pyrithione and zinc pyrithione), chlorphenesin, phenyl mercuric salts, phenoxyethanol, and other known preservative systems.

In some embodiments, the preservative includes one or more preservatives, the one or combination present at a concentration, by weight of about 0.001% to about 5%, or alternatively about 0.05% to about 2.5% or alternatively about 0.1% to about 2.0%, based upon weight of the cosmetic cleansing composition.

Thus, any one of or a combination of preservatives, when present, may be present, by weight, based on the total weight of the cosmetic cleansing composition, is from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 1.0, 1.1, 1.2, 1.3, 1.4, 1.5, 1.6, 1.7, 1.8, 1.9, 2, 3, 4, to about 5 weight percent, including increments and ranges therein and there between.

Active Compounds

In some embodiments, embodiments of the cosmetic cleansing composition may include one or more additional compounds selected from active compounds, in particular keratinous tissue actives, such as, but not limited to, skin actives. In some embodiments, optional active compounds may be selected from the group consisting of: vitamins, for example, Vitamin E, carnosine, hyaluronic acid, panthenol, at least one sun filter, at least one hydroxy acid, and combinations thereof. In some embodiments, an active compound is selected from the group consisting of alpha, beta or polyhydroxy acids selected from the group consisting of lactic acid, glycolic acid, salicylic acid, malic acid, tartaric acid, citric acid, mandelic acid, lactobionic acid, gluconolactone, galactose, and combinations thereof. For the avoidance of doubt, the cosmetic cleansing composition may include one or more of any one of the optional active compounds. For example, the cosmetic cleansing composition may include one, or more than one hydroxy acid, one, or more than one vitamin, and the like.

Vitamin E may be selected from the group consisting of alpha-tocopherol, beta-tocopherol, delta-tocopherol, gamma-tocopherol, and alpha-tocotrienol, beta-tocotrienol, delta-tocotrienol, gamma-tocotrienol, and derivatives thereof. Salts or derivatives of tocopherols include pharmaceutically acceptable compounds such as acetate, sulfate, succinate, nicotinate, palmitate, allophanate, phosphate, quinone, or halogenated derivatives, esters, or stereoisomers, for example. The invention encompasses the use of Vitamin E derivatives in which substitutions, additions, and other alterations have been made in the 6-chromanol ring and/or side chain, with the proviso that the derivatives maintain the antioxidant activity of Vitamin E. Additional tocopherols can be constructed by conjugation to the ring structure or side chain of various other moieties, such as those containing oxygen, nitrogen, sulfur and/or phosphorus. Tocopherol derivatives can also be made by modifying the length of the side chain from that found in tocopherols such as alpha-, beta-, delta- and gamma-tocopherol. Tocopherols can also vary in stereochemistry and saturation of bonds in the ring structure and side chain. Additional tocopherol derivatives, including prodrugs, can be made by conjugation of sugars or other moieties to the side chain or ring structure. Tocopherols include without limitation stereoisomers (e.g., + and − stereoisomers of alpha-tocopherol; (+/−) indicates a racemic mixture) or mixtures of structurally distinct tocopherols (e.g., alpha-plus gamma-tocopherol). Tocopherols may be obtained from Roche, Nutley, N.J., for example.

In some embodiments, additional actives may include one or more hydroxy acid selected from alpha, beta or polyhydroxy acids. Thus, in various embodiments, a hydroxy acid may be selected from the group consisting of lactic acid, glycolic acid, salicylic acid, malic acid, tartaric acid, citric acid, mandelic acid, lactobionic acid, gluconolactone, galactose, and combinations thereof.

In some embodiments, additional actives can also include one or more of the antioxidants selected from the group consisting of mangiferin, baicalin, resveratrol, tannic acid, polyphenols, amino acids and derivatives thereof, imidazoles, carnosine derivatives, carotenoids, carotenes (such as α-carotene, β-carotene, and lycopene), vitamin A, co-enzyme Q10, bioflavonoids, glutathione, plant extracts (such as rosemary extract, olive leaf extracts), green tea extracts, and combinations thereof.

In some particular embodiments, the cosmetic cleansing composition includes a combination of active compounds present in the cosmetic cleansing composition comprising tocopherol, panthenol, hyaluronic acid, hydroxy acid, carnosine, or a combination thereof.

Generally, each of the optional active compounds is present in the cosmetic cleansing composition in an amount from about 0.1% to about 30%, and in some embodiments, from about 0.5% to about 30%, and in some embodiments, from about 0.5% to about 15%, and in some embodiments, from about 0.1% to about 1%, and in some embodiments, from about 1% to about 2%, or any suitable combination, sub-combination, range, or sub-range thereof by weight, based on the weight of the cosmetic cleansing composition.

Thus, in various embodiments, optional active compounds, if present, are present in a composition according to the disclosure, and each of the individual components in the ranges as described herein above, from about 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 2, 3, 4, 5, 6, 7, 8, 9, 10, 11, 12, 13, 14, 15, 16, 17, 18, 19, 20, 21, 22, 23, 24, 25, 26, 27, 28 to about 30 percent by weight, including increments and ranges there between.

Other Additives

The cosmetic cleansing composition can also comprise at least one additive used in the cosmetics field which does not affect the properties of the cosmetic cleansing composition according to the invention, such as, emulsifiers, emollients, fragrances, pH adjusters (citric acid, sodium chloride; neutralizing or pH-adjusting agents (e.g., triethanolamine (TEA) and sodium hydroxide), and combinations thereof), other cosmetically acceptable additives, such as but not limited to, pearlescent agents, silica, and coloring materials; essential oils; fruit extracts, for example, Pyrus malus (Apple) Fruit Extract, and Aloe Barbadensis Leaf Juice Powder. Although the optional additives are given as examples, it will be appreciated that other optional components compatible with cosmetic applications known in the art may be used.

In accordance with the various embodiments, the amount of one or more additives, alone or in combination, present in the cosmetic cleansing composition can be present in the cosmetic cleansing composition according to the disclosure in a range from about 0.001% to about 20%, by weight, or from about 0.005% to about 0.01%, or from about 0.01% to about 0.1%, or from about 0.15% to about 5%, or from about 0.40% to about 4%, or from about 0.5% to about 2.5% by weight, or from about 1% to about 2%, or any suitable combination, sub-combination, range, or sub-range thereof by weight, based on the total weight of the cosmetic cleansing composition.

Thus, any one or a combination of additives may be present, by weight, based on the total weight of the cosmetic cleansing composition, each one or the combination present from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 2, 3, 4, 5, 6, 7, 8, 9, 10, 11, 12, 13, 14, 15, 16, 17, 18, 19 to about 20 weight percent, including increments and ranges therein and there between.

Although the optional additives are given as examples, it will be appreciated that other optional components compatible with cosmetic applications known in the art may be used that are suitable. It will be appreciated by a skilled artisan that any optional additives are present only to the extent and in amounts that do not materially adversely affect the basic and novel characteristic(s) of the claimed disclosure. Thus, in some embodiments that include optional additives, such optional additives will not materially adversely affect the solubility of the skin actives of the cosmetic cleansing composition. And in some embodiments that include optional additives, such optional additives will not materially adversely affect the cosmetic cleansing composition forming a single phase solution.

In some particular embodiments, the cosmetic cleansing composition may include optional additives, for example one or more of phenoxyethanol, terephthalylidene dicamphor sulfonic acid, trisodium ethylenediamine disuccinate, sodium citrate, sodium chloride, hydroxyacetophenone sodium benzoate, potassium sorbate, citric acid, caprylyl glycol, trisodium ethylenediamine disuccinate, or combinations thereof. In some embodiments, phenoxyethanol is present as a preservative in the cosmetic cleansing composition in a range from about 0.5% to about 2%, and in some embodiments in a range from about 1% to about 2%.

In accordance with the various embodiments, the amount of one or more actives and additives, alone or in combination, when present in the cosmetic cleansing composition according to the disclosure can be present in a range from about 0.001% to about 20%, and in some embodiments, from about 0.05% to about 0.01%, and in some embodiments, from about 0.01% to about 0.1%, and in some embodiments, from about 0.15% to about 5%, and in some embodiments, from about 0.40% to about 4%, and in some embodiments, from about 0.5% to about 2.5%, and in some embodiments, from about 0.1% to about 0.5%, and in some embodiments, from about 1% to about 2%, or any suitable combination, sub-combination, range, or sub-range thereof by weight, based on the total weight of the cosmetic cleansing composition. One of ordinary skill in the art, however, will appreciate that other ranges are within the scope of the invention.

Thus, any one or a combination of actives and additives may be present, each one or the combination present from about 0.001, 0.005, 0.01, 0.02, 0.03, 0.04, 0.05, 0.06, 0.07, 0.08, 0.09, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 2, 3, 4, 5, 6, 7, 8, 9, 10, 11, 12, 13, 14, 15, 16, 17, 18, 19 to about 20 percent, by weight, including increments and ranges therein and there between.

Product Forms

The cosmetic cleansing composition may be any suitable cosmetic cleansing composition, such as, but not limited to, an anhydrous cleansing oil, foaming cleanser, a cleansing milk, lotion or cream, an emulsion such as a water-in-oil or oil-in-water emulsion, or a multi-phase composition comprising at least one water phase, and is easy on the skin, efficiently removes makeup, sebum, dirt and skin debris without smudging, and provides a soft and smooth skin feel after use. In some embodiments, the cosmetic cleansing composition is free from surfactants. In some embodiments, the cosmetic cleansing composition includes one or more surfactants.

In some embodiments, the cosmetic cleansing composition is applied to keratinous tissue using an pad or wipe comprising a solid substrate, for example comprising a disposable cotton or other type pad, a cloth, a brush or other type applicator.

In some embodiments, the disclosure provides articles of manufacture that include the cosmetic cleansing composition provided in an article in the form of a pad or wipe comprising a solid substrate.

Pad or Wipe

In addition to cosmetic cleansing compositions, embodiments of the present invention may include a pad or wipe infused with the cosmetic cleansing composition according to the present invention.

In accordance with the various embodiments, provided are pads or wipes comprising a solid substrate imbued with an inventive cosmetic cleansing composition. The makeup removal and cleansing pad or wipe is formed from synthetic materials or formed from natural biodegradable and sustainably sourced natural originated fiber, natural fiber, or regenerated or recycled natural fiber.

According to some embodiments, the substrate is a nonwoven material. A general description of nonwoven materials is given in Riedel “Nonwoven Bonding Methods and Materials,” Nonwoven World (1987). These substrates are obtained according to the normal methods of the technology for the preparation of nonwoven materials. According to one embodiment, the substrate is a nonwoven material, composed of viscose or a nonwoven material composed of a viscose/microviscose mixture, through a hydro entanglement process.

When the substrate is a nonwoven material, use may be made of a nonwoven material which does not go into ball and which is sturdy enough not to disintegrate and not to become fluffy when applied to the skin. It must be absorbent and soft at least on one side for the removal of makeup from the eyes in particular. Mention may be made, as appropriate nonwoven materials, for example, of those sold under the names Ultraloft 15285-01, Ultraloft 182-008, Ultraloft 182-010 and Ultra loft 182-016 by BBA, Vilmed M1519 Blau, Vilmed M 1550 N and 112-132-3 by Freudenberg, that sold under the name Norafin 11601-010B by Jacob Holm Industries, the flocked nonwoven materials sold under the names Univel 109 and Univel 119 by Uni Flockage and that made of viscose/PLA supplied by Sandler.

According to some embodiments comprising an pad or wipe, a water insoluble substrate is employed. The water-insoluble substrate can comprise one or more layers and it can be chosen from the group consisting of woven materials, nonwoven materials, foams, sponges, waddings, as sheets, balls or films. It can in particular be a nonwoven substrate based on fibers of natural origin (flax, wool, cotton, silk, viscose, fibers made of bamboo) or synthetic origin (cellulose derivatives, polyvinyl derivatives, polyesters, such as poly(ethylene terephthalate), polyolefins, such as polyethylene (PET) or polypropylene, polyamides, such as Nylon, or acrylic derivatives) and their mixtures, such as viscose/PET, polylactic acid (PLA) or viscose/polylactic acid (viscose/PLA).

Many conventional cosmetic articles of manufacture, such as wipes, are formed of fossil-based polymeric materials, such as PET (Polyethylene terephthalate, or polyester) and PP (polypropylene), which may be used alone or in combination with natural-based material. Such synthetic substrates are desirable because they are relatively less costly to produce and deliver good tensile strength, however, because they essentially never degrade, they contribute adversely to the environment. With increasing focus in the cosmetics industry on sustainability, there is a need for cosmetic articles of manufacture that employ solid substrates to draw from sources such as (1) natural originated fiber based nonwovens, such as pulp, viscose, lyocell, cellulose acetate, cotton, (2) natural fiber based nonwovens, such as hemp, flax, seaweed, ramie, banana, pineapple, and (3) regenerated or recycled fiber based nonwovens, such as cotton. As used herein, the terms “natural originated fiber” refers to fiber materials that are formed by chemical processing that render derivatives based on natural fibers; “natural fiber” means non-derivative forms of natural fibers, and “regenerated/recycled” means natural fibers that are reclaimed from goods formed with such fibers.

In some embodiments, the pad or wipe herein is formed of substrates that are obtained from sustainable sources and are biodegradable. In some particular embodiments, these materials are selected from natural originated fiber formed into nonwovens, using fibers such as pulp, viscose, lyocell, cellulose acetate, and cotton. And in certain embodiments, the substrates used to form cosmetic pads or wipes are nonwovens of lyocell. According to those embodiments that include natural biodegradable and sustainably sourced fibers, a clear benefit realized is the opportunity to reduce the environmental burden. Other benefits may also be achieved using sustainable and biodegradable substrates. As further described herein, exemplified pads or wipes that employ sustainable and biodegradable substrates, particularly lyocell nonwovens, demonstrate unexpected mechanical, textural, and absorbent properties and significantly enhanced performance as compared with competitive cosmetic pads or wipes that employ synthetic materials. Also as described herein, exemplified pads or wipes that employ sustainable and biodegradable substrates, particularly lyocell nonwovens, demonstrate enhanced performance even as compared with inventive compositions hereof that are imbued in a synthetic substrate.

An article according to the disclosure is moist to the touch. It exhibits the advantage of being comfortable during application to the skin and having a nourishing effect due to the presence of an oily phase. When it is used for cleaning or removing makeup from the skin, an article according to the disclosure is passed over the skin, while possibly leaving it applied for a time sufficient for the makeup products to be dissolved in the impregnating composition of the article, and then the skin is wiped. The skin can also optionally be rinsed subsequently. The article according to the invention is preferably a cosmetic article appropriate for caring for and/or treating the skin of the face, body or hands and for cleaning or removing makeup from the skin of the face and/or body. It can also be used for caring for the hair and for removing makeup from the eyes.

An article according to the disclosure can have any form appropriate to the desired objective. It can constitute a wipe that has a generally rectangular, square or other shape that may be single ply, multiple ply, and may be folded or un-folded. The article can also be in the form of a glove, of a mitten or in any other form appropriate for practical use on the face or the body, for example in the form of a face with holes for the sites of the eyes, nose and/or mouth, or in the form of a makeup-removing fingerstall for application in removing makeup from the eyelashes, or in the form of a single- or double-sided disc which can in particular comprise two sides impregnated with different compositions. The article can also comprise a rough surface which makes possible the exfoliation (scrubbing) of the skin.

### Embodiments of the Invention

In some embodiments the invention provides a cosmetic cleansing composition comprising:
a) cleansing coagulant system comprising:
   i. an oil component; and
   ii. a polymer component; and
b) a cosmetic carrier system comprising one or more phases selected from the group consisting of a water phase, an oil phase, and combinations thereof.

In some embodiments of the cosmetic cleansing composition, the at least one oil in the cleansing coagulant system is selected from the group consisting of glycerides, fatty acids, alkenes, alkynes, and combinations thereof.

In some embodiments of the cosmetic cleansing composition, the at least one oil in the cleansing coagulant system is selected from the group consisting of sunflower oil, soybean oil, olive oil, corn oil, meadowfoam oil, and other natural oils.

In some embodiments of the cosmetic cleansing composition, the at least one oil in the cleansing coagulant system is selected from silicone oils.

In some embodiments of the cosmetic cleansing composition, the at least one viscosity modifier in the cleansing coagulant system is selected from the group consisting of synthetic polymers, natural polymers, and combinations thereof.

In some embodiments of the cosmetic cleansing composition, the at least one viscosity modifier in the cleansing coagulant system is selected from the group consisting of petrochemical methacrylates, acrylates, allyl stearate/VA copolymer, and combinations thereof.

In some embodiments of the cosmetic cleansing composition, the at least one viscosity modifier in the cleansing coagulant system is selected from natural polymers selected from the group consisting of xanthan gum, guar gums, k-carrageenan, locust bean gum, alkyl dextrin, silica silicate, silica, montmorillonite, dibutyl ethylhexanoyl glutamide, dibutyl lauroyl glutamide, and combinations thereof.

In some embodiments of the cosmetic cleansing composition, the at least one oil in the cleansing coagulant system is present in a range from about 30% to about 45%, by weight, based on the weight of the cleansing coagulant system.

In some embodiments of the cosmetic cleansing composition, the at least one polymer is present in a range from about 0.1% to about 20%, by weight, based on the weight of the cleansing coagulant system.

In some embodiments of the cosmetic cleansing composition, the cleansing coagulant system is present in the cosmetic cleansing composition in a range from about 1% to about 25%, by weight, based on the weight of the cosmetic cleansing composition.

In some embodiments of the cosmetic cleansing composition, the cosmetic carrier system is present in the range from about 75% to about 99%, by weight, based on the weight of the cosmetic cleansing composition.

In some embodiments of the cosmetic cleansing composition, the cosmetic carrier system is essentially anhydrous and includes only any oily phase.

In some embodiments of the cosmetic cleansing composition, one or both of the cleansing coagulant system and the cosmetic carrier system includes one or more of components selected from the group consisting of oils, oil-phase solvents, water, water soluble solvents, surfactants, humectants, thickeners, preservatives, actives, powders, fragrance oils, colorants, chelating agents, fragrances, and combinations thereof.

The cosmetic cleansing composition further comprising a cosmetic coagulant system solvent.

In some embodiments of the cosmetic cleansing composition, the solvent is present in cosmetic coagulant system, the cosmetic carrier system, or both.

In some embodiments of the cosmetic cleansing composition, the solvent comprises oil.

In some embodiments of the cosmetic cleansing composition, the solvent comprises glycerin, C1-4 alcohols, organic solvents, fatty alcohols, fatty ethers, fatty esters, polyols, glycols, vegetable oils, mineral oils, liposomes, laminar lipid materials, water, or any combinations thereof.

In some embodiments of the cosmetic cleansing composition, the solvent comprises a cosmetically acceptable organic solvent selected from ethylene glycol, propylene glycol, butylene glycol, hexylene glycol, propane diol, glycerin, or any combinations thereof.

The cosmetic cleansing composition according to claim 1, wherein, when the cosmetic cleansing composition is applied to a keratinous tissue, for example skin or artificial skin that is soiled with one or more of sebum, makeup, or dirt and the % sebum, makeup or dirt extracted is quantified, significant sebum/dirt/oil containing makeup is removed from the skin.

In some embodiments of the cosmetic cleansing composition, the cosmetic cleansing composition is a product form selected from the group consisting of an anhydrous cleansing oil, a foaming cleanser, a cleansing milk, a cleansing lotion, a cleansing cream, a water-in-oil emulsion, an oil-in-water emulsion, a multi-phase composition comprising at least one water phase.

In some embodiments of the cosmetic cleansing composition, the cosmetic cleansing composition is free from surfactants.

In some embodiments of the cosmetic cleansing composition, the cosmetic cleansing composition is one or more of a low water, water free, leave-on, a peel off, or a rinse-off formulation.

In some embodiments of the cosmetic cleansing composition, the cosmetic cleansing composition is applied to keratinous tissue using an article comprising a solid substrate selected from a disposable pad, a cloth, a brush, or a cosmetic applicator.

In some embodiments of the cosmetic cleansing composition, the cleansing coagulant system is formed just prior to or at the time of application to the keratinous tissue.

In some embodiments of the cosmetic cleansing composition, cleansing coagulant system forms as a film or mask on application that peels away in one piece or more than one piece from the skin.

In some embodiments of the cosmetic cleansing composition, the cosmetic cleansing composition sets or dries into a film or mask which can be peeled away or sloughed off in one piece in less than 10 minutes.

In some embodiments of the cosmetic cleansing composition, minimal or no water is needed for utilizing the cosmetic cleansing composition.

In some embodiments of the cosmetic cleansing composition, no water is required for use or removal of the cosmetic cleansing composition.

In some embodiments of the cosmetic cleansing composition, the cleansing coagulant system is included in the cosmetic cleansing composition in an activated form with or without a solvent.

In some embodiments of the cosmetic cleansing composition, the cosmetic cleansing composition includes a solvent that is present in the cleansing coagulant system, the cosmetic carrier system, or both.

In some embodiments of the cosmetic cleansing composition, the cosmetic cleansing composition includes a cosmetic carrier system comprising one or more phases selected from the group consisting of a water phase, an oil phase, and combinations thereof.

In some embodiments of the cosmetic cleansing composition, cosmetic cleansing composition is essentially anhydrous and includes only any oily phase.

In some embodiments of the cosmetic cleansing composition includes an article of manufacture that includes the cosmetic cleansing composition provided in a solid form, imbued in a pad or a wipe, or contained in a pod or a packet.

In some embodiments of the cosmetic cleansing composition includes the cosmetic cleansing composition is provided with an article of manufacture comprising a pad or a wipe formed from synthetic materials or formed from natural biodegradable and sustainably sourced natural originated fiber, natural fiber, or regenerated or recycled natural fiber.

In some embodiments of the cosmetic cleansing composition includes the article of manufacture is a pad or a wipe infused with one or both of the cleansing coagulant system and the cosmetic carrier system.

In some embodiments of the cosmetic cleansing composition, the cosmetic cleansing composition includes an article of manufacture that includes the cosmetic cleansing composition provided in at least one pod or packet that contains the cleansing coagulant system.

In some embodiments of the cosmetic cleansing composition includes an article of manufacture that includes both the cleansing coagulant system and the cosmetic carrier system.

In some embodiments of the cosmetic cleansing composition includes an article of manufacture that includes the cleansing coagulant system alone or with a solvent, and the article of manufacture includes at least one other article selected from a pod, packet or wipe that contains one of a cleansing coagulant system activator, the cosmetic carrier system, a cleansing coagulant system solvent, or a combination thereof.

In some embodiments of the cosmetic cleansing composition, cosmetic cleansing composition is an essentially anhydrous oil based composition that includes the cleansing coagulant system alone or with a solvent imbued or contained in an article comprising one of a pad, wipe, pod or packet, wherein the cosmetic carrier is either contained with the cleansing coagulant system or is separately imbued or contained in an article comprising one of a pad, wipe, pod or packet and comprises one of a cleansing coagulant system activator, a cleansing coagulant system solvent, one or more oils, one more ore oil-soluble components, or a combination thereof.

In some embodiments in use, when the cosmetic cleansing composition is applied to a keratinous tissue, for example skin or artificial skin that is soiled with one or more of sebum, makeup, or dirt and the % sebum, makeup or dirt extracted is quantified, significant sebum/dirt/oil containing makeup will be removed from the skin.

In some embodiments of the cosmetic cleansing composition, the cosmetic cleansing composition is a leave-on, wipe off, or a rinse-off formulation.

In some embodiments of the cosmetic cleansing composition, the cosmetic cleansing composition is applied to keratinous tissue using an article comprising a solid substrate, for example comprising a disposable cotton or other type pad, a cloth, a brush or other type applicator.

In some embodiments of the cosmetic cleansing composition, the cosmetic cleansing composition is applied to keratinous tissue in a generally liquidous form and thereupon forms into a solid or semi solid or film that may be peeled or sloughed off to remove makeup, sebum, soil and the like.

In some embodiments of the cosmetic cleansing composition, the cosmetic cleansing composition is applied in a series of steps from one or more articles of manufacture wherein after a first application one or more subsequent applications provides for reacting, fixing or coagulating the cleansing coagulant system component to form a semi solid or solid or congealed material that may thereafter be peeled or sloughed of washed form the keratinous tissue.

In some embodiments the invention provides an article of manufacture, comprising: a solid substrate formed from one or a combination of synthetic materials and natural biodegradable fiber imbued with a cleansing composition comprising:

cleansing coagulant system comprising an oil component; and a polymer component; and a cosmetic carrier system comprising one or more phases selected from the group consisting of a water phase, an oil phase, and combinations thereof.

In some embodiments the invention provides an article of manufacture, comprising: at least one solid component comprising a pod that comprises a water soluble encapsulating film, a water insoluble packet, a fibrous substrate, or a combination thereof, and a cleansing composition comprising: a cleansing coagulant system comprising: an oil component; and a polymer component; and a cosmetic carrier system comprising one or more phases selected from the group consisting of a water phase, an oil phase, and combinations thereof, wherein, each of the cleansing coagulant system and the cosmetic carrier system are one of: contained separately in discrete solid components; or contained together in a single solid component.

In some embodiments the invention provides a method for cleansing skin, comprising: providing a cosmetic cleansing composition according to any one of the foregoing described embodiments or combinations thereof, wherein (i) the cosmetic cleansing composition is applied to keratinous tissue using an article comprising a solid substrate selected from a disposable pad, a cloth, a brush, or a cosmetic applicator, or wherein the cosmetic cleansing composition is applied to keratinous tissue in a generally liquidous form.

In some embodiments of the method for cleansing skin, the cleansing coagulant system is formed just prior to or at the time of application to the keratinous tissue, and wherein the cleansing coagulant system forms as a film or mask on application that peels away in one piece or more than one piece from the skin.

In some embodiments of the method for cleansing skin, the cosmetic cleansing composition sets or dries into a film or mask which can be peeled away or sloughed off in one piece in less than 10 minutes, and wherein, when the cosmetic cleansing composition is applied to a keratinous tissue, for example skin or artificial skin that is soiled with one or more of sebum, makeup, or dirt and the amount of sebum, makeup or dirt extracted is quantified by weight based on the weight of the applied composition, significant sebum/dirt/oil containing makeup is removed from the skin.

In some embodiments of the method for cleansing skin, the cosmetic cleansing composition is applied in a series of steps from one or more articles of manufacture wherein after a first application one or more subsequent applications provides for reacting, fixing or coagulating the cleansing coagulant system component to form a semi solid or solid or congealed material that may thereafter be peeled or sloughed of washed form the keratinous tissue.

In some embodiments of the method for cleansing skin, minimal or no water is needed for utilizing the cosmetic cleansing composition.

According to the disclosure, the inventive compositions overcome the shortcomings of the prior art and provide unexpected benefits that include ease with overall makeup removal, easy foundation removal, leaves skin feeling clean and fresh, soft and smooth, easy mascara removal (regular and waterproof) with no rubbing, satisfying size and thickness of substrate (wipe), not too dry, non-greasy, does not leave a residue and pleasant fragrance.

The terms “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“One or more,” as used herein, means at least one, and thus includes individual components as well as mixtures/combinations.

The transitional terms “comprising,” “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All materials and methods described herein that embody the present invention can, in alternate embodiments, be more specifically defined by any of the transitional terms “comprising,” “consisting essentially of,” and “consisting of.”

Other than in the operating examples, or where otherwise indicated, all numbers expressing quantities of ingredients and/or reaction conditions are to be understood as being modified in all instances by the term “about,” meaning within 10% of the indicated number (e.g., “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

While the invention has been described with reference to a preferred embodiment, it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention is not limited to the particular embodiment disclosed as the best mode contemplated for carrying out this invention, but that the invention will include all embodiments falling within the scope of the appended claims.

What I claim:

1. A cosmetic cleansing composition comprising:
a) cleansing coagulant system comprising:
i. an oil component; and
ii. a polymer component; and
b) a cosmetic carrier system comprising one or more phases selected from the group consisting of a water phase, an oil phase, and combinations thereof.

2. The cosmetic cleansing composition according to claim 1, wherein the at least one oil in the cleansing coagulant system is selected from the group consisting of glycerides, fatty acids, alkenes, alkynes, and combinations thereof, and wherein the at least one viscosity modifier in the cleansing coagulant system is selected from the group consisting of synthetic polymers, natural polymers, and combinations thereof.

3. The cosmetic cleansing composition according to claim 1, wherein the at least one oil in the cleansing coagulant system is selected from the group consisting of sunflower oil, soybean oil, olive oil, corn oil, meadowfoam oil, silicone oil, and combinations thereof, and wherein the at least one viscosity modifier in the cleansing coagulant system is selected from the group consisting of petrochemical methacrylates, acrylates, allyl stearate/VA copolymer, xanthan gum, guar gums, k-carrageenan, locust bean gum, alkyl dextrin, silica silicate, silica, montmorillonite, dibutyl ethylhexanoyl glutamide, dibutyl lauroyl glutamide, and combinations thereof.

4. The cosmetic cleansing composition according to claim 1, wherein the at least one oil in the cleansing coagulant system is present in a range from about 30% to about 45%, by weight, based on the weight of the cleansing coagulant system, and wherein the at least one polymer is present in a range from about 0.1% to about 20%, by weight, based on the weight of the cleansing coagulant system, and wherein the cleansing coagulant system is present in the cosmetic cleansing composition in a range from about 1% to about 25%, by weight, based on the weight of the cosmetic cleansing composition, and wherein the cosmetic carrier system is present in the range from about 75% to about 99%, by weight, based on the weight of the cosmetic cleansing composition.

5. The cosmetic cleansing composition according to claim 1, wherein one or both of the cleansing coagulant system and the cosmetic carrier system includes one or more of components selected from the group consisting of oils, oil-phase solvents, water, water soluble solvents, surfactants, humectants, thickeners, preservatives, actives, powders, fragrance oils, colorants, chelating agents, fragrances, and combinations thereof.

6. The cosmetic cleansing composition according to claim 1, further comprising a cosmetic coagulant system solvent, wherein the solvent is present in cosmetic coagulant system, the cosmetic carrier system, or both, and wherein the solvent is selected from the group consisting of water, ethylene glycol, propylene glycol, butylene glycol, hexylene glycol, propane diol, glycerin, and combinations thereof.

7. The cosmetic cleansing composition according to claim 1, wherein the cosmetic cleansing composition is a product form selected from the group consisting of an anhydrous cleansing oil, a foaming cleanser, a cleansing milk, a cleansing lotion, a cleansing cream, a water-in-oil emulsion, an oil-in-water emulsion, a multi-phase composition comprising at least one water phase.

8. The cosmetic cleansing composition according to claim 1, wherein the cosmetic cleansing composition is one of a leave-on, a peel off, or a rinse-off formulation.

9. The cosmetic cleansing composition according to claim 1, wherein the cosmetic cleansing composition is free from surfactants.

10. The cosmetic cleansing composition according to claim 1, wherein the cosmetic cleansing composition includes a solvent that is present in the cleansing coagulant system, the cosmetic carrier system, or both.

11. The cosmetic cleansing composition according to claim 1, wherein the cosmetic cleansing composition includes a cosmetic carrier system comprising one or more phases selected from the group consisting of a water phase, an oil phase, and combinations thereof.

12. The cosmetic cleansing composition according to claim 1, comprising an article of manufacture characterized by one or more features selected from the group consisting of (i) an article of manufacture that includes the cosmetic cleansing composition provided in a solid form, imbued in a pad or a wipe, or contained in a pod or a packet, (ii) an article of manufacture comprising a pad or a wipe formed from synthetic materials or formed from natural biodegradable and sustainably sourced natural originated fiber, natural fiber, or regenerated or recycled natural fiber, (iii) an article of manufacture that includes a pad or a wipe infused with one or both of the cleansing coagulant system and the cosmetic carrier system, (iv) an article of manufacture that includes the cosmetic cleansing composition provided in at least one pod or packet that contains the cleansing coagulant system, (v) an article of manufacture that includes both the cleansing coagulant system and the cosmetic carrier system, (vi) an article of manufacture that includes the cleansing coagulant system alone or with a solvent, and the article of manufacture includes at least one other article selected from a pod, packet or wipe that contains one of a cleansing coagulant system activator, the cosmetic carrier system, a cleansing coagulant system solvent, or a combination thereof, and (vii) a combination thereof.

13. The cosmetic cleansing composition according to claim 1, wherein cosmetic cleansing composition is an essentially anhydrous oil based composition that includes the cleansing coagulant system alone or with a solvent imbued or contained in an article comprising one of a pad, wipe, pod or packet,

14. The cosmetic cleansing composition according to claim 1, wherein the cosmetic carrier is either contained with the cleansing coagulant system or is separately imbued or contained in an article comprising one of a pad, wipe, pod, or packet and comprises one of a cleansing coagulant system activator, a cleansing coagulant system solvent, one or more oils, one more ore oil-soluble components, or a combination thereof.

15. A method for cleansing skin, comprising: providing a cosmetic cleansing composition according to claim 1, wherein (i) the cosmetic cleansing composition is applied to keratinous tissue using an article comprising a solid substrate selected from a disposable pad, a cloth, a brush, or a cosmetic applicator, or wherein the cosmetic cleansing composition is applied to keratinous tissue in a generally liquidous form.

16. The method for cleansing skin according to claim 15, wherein the cleansing coagulant system is formed just prior to or at the time of application to the keratinous tissue, and wherein the cleansing coagulant system forms as a film or mask on application that peels away in one piece or more than one piece from the skin.

17. The method for cleansing skin according to claim 15, wherein the cosmetic cleansing composition sets or dries into a film or mask which can be peeled away or sloughed off in one piece in less than 10 minutes, and wherein, when the cosmetic cleansing composition is applied to a keratinous tissue, for example skin or artificial skin that is soiled with one or more of sebum, makeup, or dirt and the amount of sebum, makeup or dirt extracted is quantified by weight based on the weight of the applied composition, significant sebum/dirt/oil containing makeup is removed from the skin.

18. The method for cleansing skin according to claim 15, wherein the cosmetic cleansing composition is applied in a series of steps from one or more articles of manufacture wherein after a first application one or more subsequent applications provides for reacting, fixing or coagulating the cleansing coagulant system component to form a semi solid or solid or congealed material that may thereafter be peeled or sloughed of washed form the keratinous tissue.

19. The method for cleansing skin according to claim 15, wherein minimal or no water is needed for utilizing the cosmetic cleansing composition.

20. An article of manufacture, comprising:
at least one component selected from the group consisting of:
(A) a solid substrate component formed from one or a combination of synthetic materials and natural biodegradable fiber imbued with a cleansing composition comprising:
a) cleansing coagulant system comprising:
i. an oil component; and
ii. a polymer component; and
b) a cosmetic carrier system comprising one or more phases selected from the group consisting of a water phase, an oil phase, and combinations thereof;
(B) at least one solid component comprising a pod that comprises a water soluble encapsulating film, a water insoluble packet, a fibrous substrate, or a combination thereof, and a cleansing composition comprising:
a) cleansing coagulant system comprising:
i. an oil component; and
ii. a polymer component; and
b) a cosmetic carrier system comprising one or more phases selected from the group consisting of a water phase, an oil phase, and combinations thereof,
wherein, each of the cleansing coagulant system and the cosmetic carrier system are one of:
contained separately in discrete solid components;
contained together in a single solid component; and
(C) a combination thereof.
